(19) United States
(12) Patent Application Publication
(10) Pub. No.: US 2026/0108724 A1
(43) Pub. Date: Apr. 23, 2026

(54) MEDICAL DEVICE FOR TARGETED TRANS-URETHRAL MUSCLE STIMULATION AND RESPONSE

(71) Applicant: Genevi Technologies, Inc., Wayland, MA (US)

(72) Inventors: Tracy Accardi, Boston, MA (US); Adam Mascari, Wayland, MA (US); Peter Rosenblatt, Delray Beach, FL (US); George Russo, Bluffton, SC (US)

(73) Assignee: Genevi Technologies, Inc., Wayland, MA (US)

(21) Appl. No.: 19/365,501

(22) Filed: Oct. 22, 2025

Related U.S. Application Data
(60) Provisional application No. 63/710,891, filed on Oct. 23, 2024.

(51) Int. Cl. A61N 1/05

(52) U.S. Cl. CPC A61N 1/0524

(57) ABSTRACT

A transvaginal stimulation device incorporates various securing mechanisms configured to maintain secure engagement between the treatment portion of a transvaginal stimulation device and target tissue to increase the efficacy of therapeutic stimulation treatment. The device includes a concave portion configured with a stimulation array of electrodes, and the securing mechanism maximizes engagement between the stimulation array and the tissue to be treated. The device includes a flexible mechanism that increases the ability of the device to conform to the vaginal canal to maximize electrode contact. The securing mechanisms may be manually controlled or automatically controlled using predictive models trained using patient data. The predictive models may include machine learning models trained to dynamically modify device position and/or therapy in response to receipt of real-time information.

### RELATED APPLICATIONS

This application claims priority to and/or receives benefit from provisional patent application 63/710,891, filed Oct. 23, 2024, titled MEDICAL DEVICE CONFIGURED FOR TARGETED TRANS-URETHRAL MUSCLE STIMULATION AND RESPONSE. The provisional patent application is hereby incorporated by reference in its entirety.

### FIELD OF THE INVENTION

The disclosure relates to the field of medical devices and more particularly to systems, devices and methods for treating urinary incontinence.

### BACKGROUND

Approximately one in four women suffer from urinary incontinence. Urinary incontinence in females can be caused by a variety of factors, including physical conditions, lifestyle choices, and medical conditions. The most common types are stress urinary incontinence (SUI) (leakage due to physical pressure on the bladder) and urge urinary incontinence (UUI) (leakage due to a sudden, uncontrollable urge to urinate).

SUI is the involuntary leakage of urine that occurs when sudden pressure is placed on the bladder, such as when coughing, laughing, sneezing, or exercising. Common causes include childbirth, being overweight, chronic coughing, nerve injuries, and surgery. Conventional treatments for SUI ranged from Kegel exercises and behavioral changes to pelvic floor physical therapy.

Various approaches have been developed to stimulate the women's pelvic floor muscle and nerve structure to treat disorders such as stress urinary incontinence. Some approaches target the entire pelvic floor muscular structure and include large transvaginal probes that do not discriminate muscle structures at or around the women's urethra. As smooth and striated muscle fibers become weak due to several reasons, for example, childbirth, it is more difficult for typical muscle stimulation to effectively improve sphincter capacity and therefore positively impact urinary incontinence disorders.

### SUMMARY OF THE INVENTION

According to one aspect, a device includes an elongate body extending axially from a proximal end to a distal end, the elongate body comprising a concave portion curved about a longitudinal axis of the elongate body. The device further includes a plurality of electrode pairs, each pair disposed at locations on the concave portion of the elongate body, where each electrode of the pair is positioned laterally from, and on opposing sides of, the longitudinal axis of the elongate body. A securing mechanism is included in the device that applies at least one of a lateral or an axial force to a wall of a vaginal canal to conform the concave portion of the elongate body about a treatment location, wherein applying force to the wall of the vaginal canal increases the contact between the plurality of electrode pairs and the treatment location.

According to further aspects, at least a one electrode pair is disposed at locations in a range of 0 to 45 degrees offset from the longitudinal axis of the elongate body. The plurality of electrode pairs may be programmed to support at least thirty-four (34) muscle stimulation therapy plans. Wireless communication may be used to select a therapy plan. The concave portion comprises a diameter between 5 mm and 10 mm to mate with the profile of the mid-urethral sphincter.

The securing mechanism may comprise a pair of longitudinal wing extensions, coupled to the proximal end of the housing and extending towards the distal end of the elongate body, wherein the wings comprise a memory shaped alloy compressible to align the wings with the elongate body for insertion into the vaginal canal, and releasable following insertion to open the wings to apply lateral forces to the vaginal wall to pull the treatment location towards the concave portion of the elongate body and to secure the device within the vaginal canal.

Alternatively, the securing mechanism may comprise an anterior lip extending from the proximal end of the elongate body, the anterior lip directed toward the anterior vaginal wall when inserted into a vaginal canal of a patient and configured to preclude insertion of the concave portion of the housing past the treatment location and a posterior lip extending from the proximal end of the elongate body, the posterior lip directed towards a posterior vaginal wall and configured to reduce rotation of the elongate body following placement of the elongate body within the vaginal canal.

Alternatively, the securing mechanism may comprise a mechanical lever comprising at least one of a spring-loaded or electrically controlled mechanism that exerts pressure laterally or axially within the vaginal canal. Alternatively, the securing mechanism may comprise a balloon which may take many shapes.

The securing mechanism may be controlled manually, wirelessly, or via a processor within the device.

With such an arrangement securing mechanisms ensure a secure fit between the transvaginal stimulation device and the area to be treated throughout treatment, conforming the concave treatment portion of the device to the treatment location and thereby increasing treatment efficacy.

According to another aspect, a device includes a disposable elongate body extending axially from a proximal end to a distal end, the elongate body comprising a concave portion curved about a longitudinal axis of the elongate body, a plurality of electrode pairs, each pair disposed at locations on the concave portion of the elongate body, at least one sensor and a controller, coupled to the electrode array, the controller activating individual electrode pairs within the electrode array according to a therapy plan for a patient. The plurality of electrodes may form an electrode array having at least one row and at least two columns, and at least a subset of the electrodes is disposed at locations on the concave surface of the housing that are between 0 and 45 degrees offset from the longitudinal axis of the elongate body. In some embodiments, the electrodes are disposed at locations on the concave surface that are between 10 and 20 degrees offset from the longitudinal axis of the elongate body. In some embodiments, the electrodes are disposed at locations on the concave surface that are 18 degrees offset from the longitudinal axis of the elongate body.

The plurality of electrodes may be programmed to support at least thirty-four (34) muscle stimulation therapy plans. The controller may update the treatment plan in response to feedback and modify the order of activation of electrodes of the electrode array in accordance with the updated treatment plan. The feedback may include sensor feedback, patient feedback, and internal feedback, including trigger detection.

With such an arrangement, a flexible therapy device is provided capable of administering 34 individual therapies to support a wide patient population range with a single device. Because the device may be operated with feedback from a user, obtained directly or via real-time sensor results, therapy may be tailored to the comfort needs of each individual. Securing mechanisms increase the efficacy of such treatment.

According to a further aspect, a device includes an elongate body extending axially from a proximal end to a distal end, the elongate body comprising a concave portion curved about a longitudinal axis of the elongate body, a plurality of electrode pairs, each pair disposed at locations on the concave portion of the elongate body, a securing mechanism that applies at least one of a lateral or an axial force to a wall of a vaginal canal to conform the concave portion of the elongate body about a treatment location, at least one sensor configured to receive, in real-time, sensor data including at least one of patient biological data and positional data and a controller, coupled to the electrode array at the at least one sensor. The controller actuates the securing mechanism in response to received sensor data and activates individual electrode pairs within the electrode array using a therapy plan based on the sensor data.

The concave portion of the housing may comprise a curvature that is selected to hug and compress an anterior vaginal wall adjacent to the urethral sphincter.

The at least one sensor may include a biological sensor selected from a group including an impedance sensor, a muscle response sensor (EMG), a temperature sensor, a blood oxygen sensor and a flow sensor, that least one sensor configured to capture biological data in real-time. The at least one sensor may be disposed on the distal portion of the housing of the elongate body and/or on the concave portion of the housing.

The at least one sensor may be a positional sensor selected from a group including an accelerometer and a gyroscope.

The controller may be removable from the housing and may include a flexible printed circuit board.

The securing mechanism may be selected from a group of expandable mechanisms including memory alloy expandable mechanisms, mechanical expandable mechanisms, resilient expandable mechanisms and fluid. The securing mechanism may comprise a mechanical lever comprising at least one of a spring-loaded or electrically controlled mechanism that exerts pressure against the anterior vaginal wall to improve contact between the concave portion of the housing and the treatment location.

The controller further may include a predictive model, trained to dynamically control at least one of the therapy plans or a position of the device in response to patient biological and positional data received in real-time. The controller may comprise a memory for storing a trained machine learning model configured to control dynamic processing of sensor data in real-time to modify at least one of the therapy plan or a position of the device in real-time to customize patient treatment.

The device may further include a communication unit configured to receive external communications, and wherein the predictive model uses the external communications to control at least one of the therapy plan or a position of the device.

With such an arrangement, an intelligent transvaginal stimulation device capable of dynamically modifying a therapy plan and/or a device position based on user and sensor feedback is provided. Such a device, that incorporates securing mechanisms to maintain engagement with the treatment location and uses sensor data to monitor the device environment, enables dynamic modification of therapies in real-time, thereby increasing the safety, efficacy and comfort of the procedure.

### BRIEF DESCRIPTION OF THE FIGURES

FIGS. 1A and 1B are anatomical illustrations of the periurethral anatomy of a female patient;

FIG. 2 illustrates an exemplary embodiment of a transvaginal stimulation device for treating stress urinary incontinence according to one aspect of the invention;

FIGS. 3A and 3B illustrate an embodiment of a transvaginal stimulation device including laterally disposed wings for securing the device at the desired location within the vaginal canal;

FIG. 4 is an exploded view of the transvaginal stimulation device of FIG. 2, illustrating the disposable and reusable components of the device;

FIG. 5 is cross-section view of the transvaginal probe of FIG. 4 taken along line A-A′;

FIG. 6 is an exemplary embodiment of a transvaginal stimulation device for treating stress urinary incontinence including sensors;

FIG. 7 is a data flow block diagram illustrating the transition from the analog to digital domain when monitoring biological and positional signals for use in real-time dynamic therapy adaptation;

FIG. 8 illustrates exemplary functional components that may be included on a printed circuit board that may be housed within the transvaginal stimulation devices disclosed herein;

FIGS. 9A and 9B illustrate the anatomical placement of a transvaginal stimulation device as described herein;

FIG. 10 is a data flow diagram illustrating the real-time dataflow that is incorporated into dynamic treatment of a urethra;

FIGS. 11A and 11B illustrate generic securing mechanisms that may be included within a transvaginal stimulation device according to one aspect of the invention;

FIGS. 12A and 12B illustrate the use of a spring-loaded securing mechanism that may be incorporated within a transvaginal stimulation device;

FIGS. 13A and 13B illustrate the use of a mechanically linked securing mechanism that may be incorporated within a transvaginal stimulation device;

FIGS. 14A and 14B illustrate the use of an inflatable member securing mechanism that may be incorporated within a transvaginal stimulation device;

FIG. 15 is a side view diagram of an exemplary transvaginal stimulation device, wherein the flexible portion of the housing extends posteriorly to secure the concave portion of the device at the appropriate location in the vaginal canal;

FIGS. 16A-16D illustrates an exemplary subset of inflatable balloon structures that may be disposed within the housing of the transvaginal stimulation device to enact different forces on the vaginal canal during expansion;

FIG. 17 illustrates an alternative embodiment of the proximal end of a transvaginal stimulation device of the invention, including an anterior and posterior lip designed to position an electrode array disposed on the concave surface at a target treatment site without shifting or rotation of the device;

FIG. 18 is a flow diagram illustrating various operations that may be undertaken to train a machine learning model for deployment in the transvaginal stimulation devices disclosed herein; and

FIG. 19 is a flow diagram outlining a process used by a transvaginal stimulation device to intelligently deliver therapy to a patient.

### DETAILED DESCRIPTION

Definitions: For the purposes of this disclosure, the below terms shall have the following meaning:

“Distal” shall be described relative to the longitudinal axis of the transvaginal stimulation device and shall indicate the end portion of the device that is closest to the cranial wall of the vagina along the longitudinal axis of the device when the device is inserted into the patient.

“Proximal” shall be described relative to the longitudinal axis of the transvaginal stimulation device and shall indicate the end portion of the device that is closest to the meatus of the urethra when the device is inserted into the patient.

“Anterior vaginal wall” shall mean the front wall of the vagina, located between the bladder and the urethra.

An improved transvaginal stimulation device is disclosed that advantageously includes one or more securing mechanisms configured to position and secure a treatment portion of a transvaginal stimulation device (TSD) to a desired treatment location within the vaginal canal. Following insertion, the securing mechanism maintains reliable contact between the stimulation electrodes and targeted tissue to improve treatment efficacy. With such an arrangement, the present invention overcomes the problems of prior art slippage of electrode devices during application of stimulation therapy.

According to one aspect, the inventors have determined that applying lateral pressure to the vaginal walls results in anterior displacement of the device within the vaginal canal. Accordingly, the various embodiments of securing mechanisms disclosed herein include one or more mechanisms that cause the TSD to exert lateral or other pressure against the vaginal walls to pull the urethral wall posteriorly towards the curved portion of the TSD.

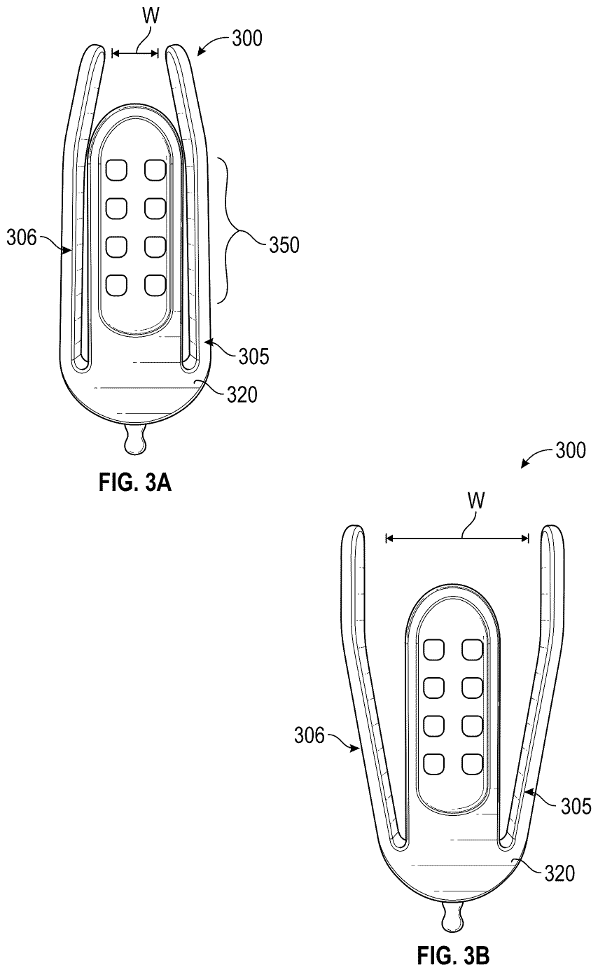

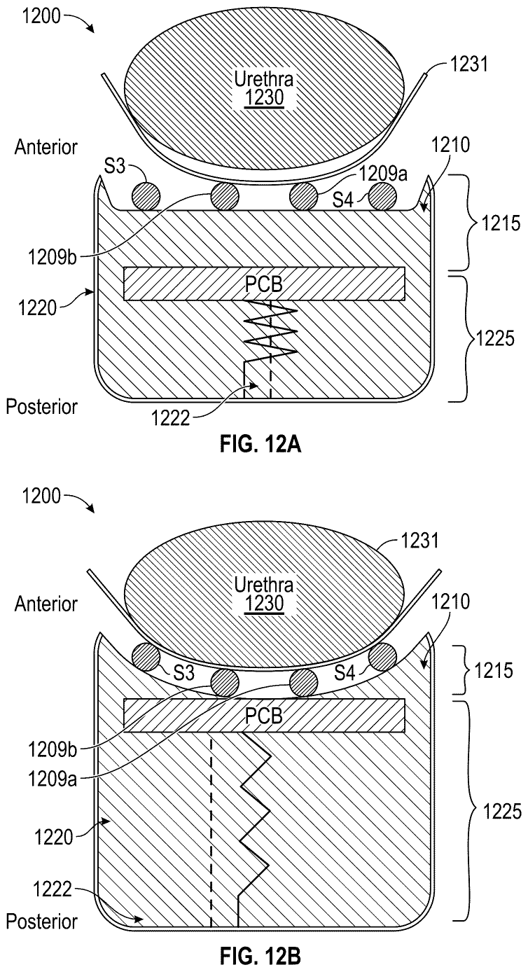

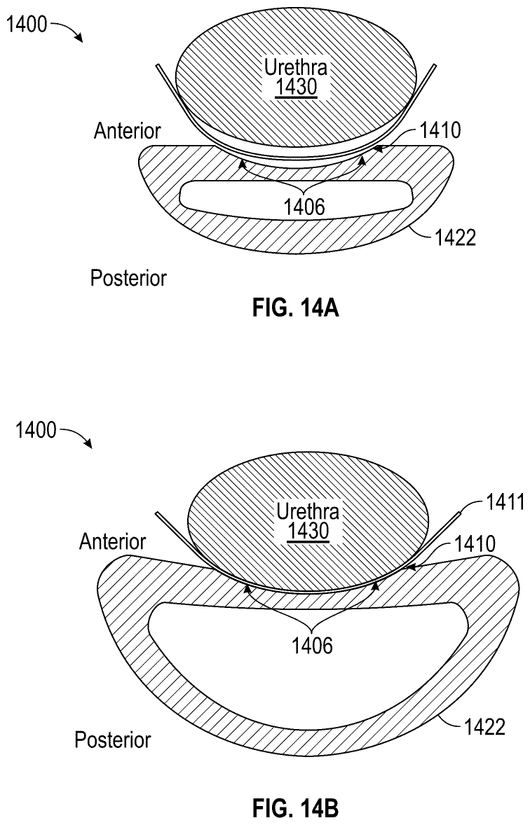

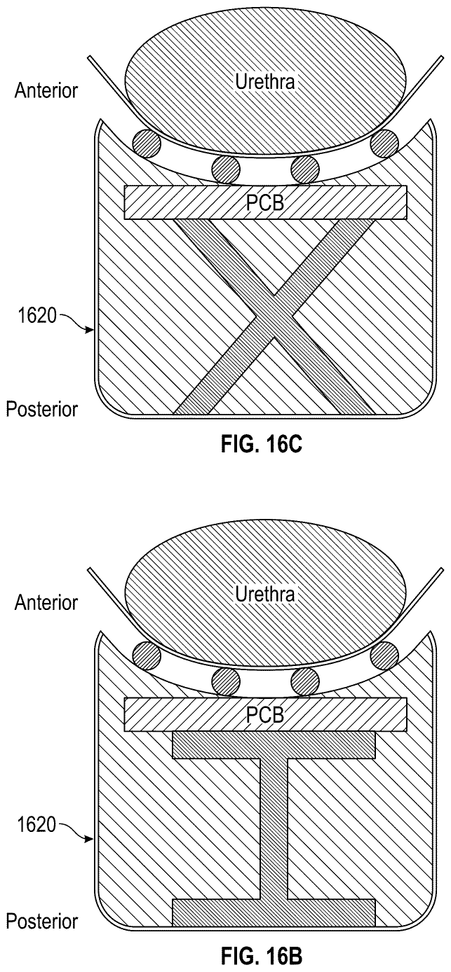

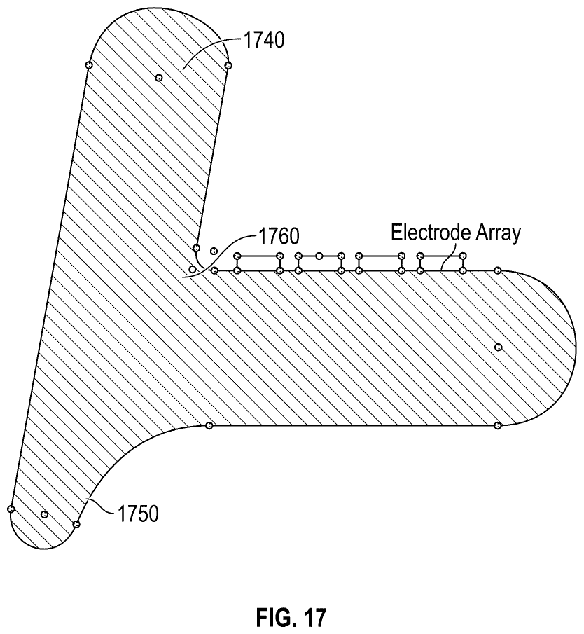

The securing mechanism may include any one of a variety of flexible elements, described herein, that expand one or more portions of the TSD 200 within the vaginal canal to increase contact pressure between the stimulation electrodes and target tissue. For example, the securing mechanisms may include features of the housing, such as a concave treatment portion, integrated wings (FIG. 3), internal lever and spring mechanisms (FIGS. 12A, 12B 13A, 13B) and/or internal inflation mechanisms (FIG. 14A, 14B, FIGS. 16A-16D), each of which is described in more detail below.

In some embodiments, the concave treatment portion is matched in diameter to mate with a treatment location, wherein an array of electrodes is disposed on the concave portion at locations selected to secure the placement of electrodes on the mid-urethral tissue. The electrode array allows a variety of patient therapy plans to be provided by a single TSD, thereby expanding the patient population pool that may benefit from the use of the device.

According to another aspect, the transvaginal stimulation device disclosed herein may include both reusable and disposable components. For example, a TSD system may include a housing including treatment electrodes, and a removable controller, storing intelligence and processing capability to manage electrode stimulation for the variety of therapy plans that may be administered by the electrodes.

According to a further aspect, the transvaginal stimulation device disclosed herein may incorporate sensors, mounted on the housing or internally to the controller. The sensors may provide both biological and positional information that may be used to select from available treatment plans for a given patient. In one embodiment, the controller may include a predictive model, that may be used to react to real-time biological or positional information to perform one of dynamically altering a therapy plan or dynamically modifying a position of the TSD within the canal in response to received biological or positional information.

In some embodiments, the predictive model may be an artificial intelligence model, trained using patient data and capable of providing real-time, personalized therapy based on received sensor data.

These and other aspects are described in detail below.

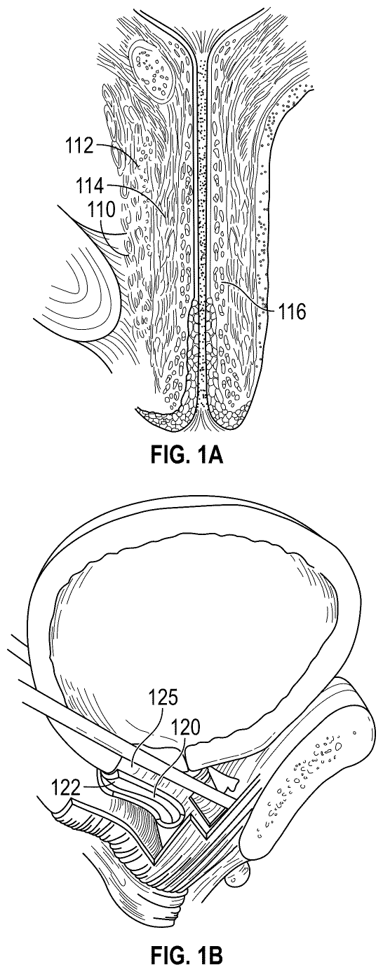

FIGS. 1A and 1B are anatomical illustrations of the periurethral anatomy of a female patient 110, provided for ease of reference when describing placement and operation of a TSD of this disclosure. FIG. 1A shows a detrusor smooth muscle at the neck of the bladder 112 and the urethra 114, itself. The urethra 114 is approximately 4-5 cm long in a female, and in FIG. 1B is shown embedded in the connective tissue 125 supporting the anterior wall 122 of the vagina 120, as shown in FIG. 1B. The urethra 114 comprises a urethral sphincter 116, which extends lengthwise along the caudal ⅘ths of the urethra 114 to actively control the flow of urine from the bladder 112, and a nozzle, which extends lengthwise along the remaining cranial ⅕th of the urethra 114 to passively control the flow of urine from the bladder 112. The urethral sphincter 116 comprises three layers of muscle (an outer circumferential layer of striated muscle 111, a middle circumferential layer of smooth muscle 112, and an inner longitudinal layer of smooth muscle 114) and a vascular plexus surrounding the urethral lumen. The circumferential striated muscle 111 is under voluntary control, while the circumferential smooth muscle 112 and longitudinal smooth muscle 114 are under involuntary or autonomic control. The circumferential striated muscle 111 contributes to the voluntary and reflex closure of the urethra 114 during acute instances that result in increased abdominal pressure (e.g., coughing, sneezing, laughing, etc.).

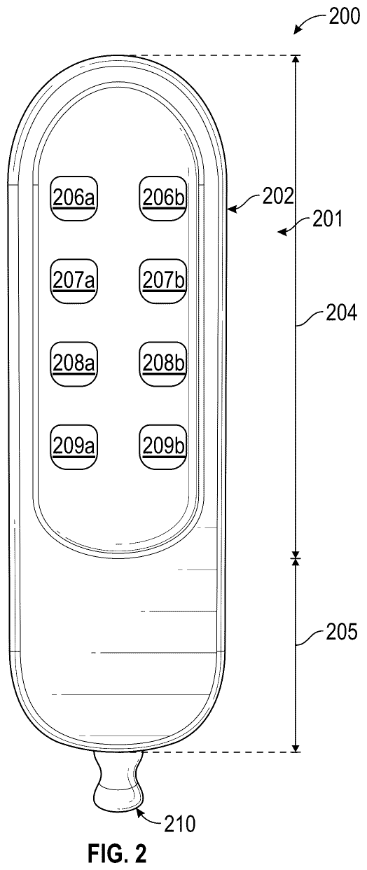

FIG. 2 illustrates an exemplary embodiment of a TSD 200 configured to perform muscle stimulation directly to the mid-urethra through the application of targeted electronic stimulation. FIG. 2 showcases the electrodes positioned for targeted stimulation at various locations along an elongate body of the TSD, highlighting the concave shape of the device designed to fit the urethral area. The elongate body 201 includes a housing 202 which may range in total length up to 10 cm, to enable the housing to rest comfortably in the distal section of the vagina. The housing may be formed from materials compatible with vaginal tissue and compliant with ISO 10993. The housing includes a first surface having a distal concave portion 204 that curves about a longitudinal axis of the elongate body and is shaped to match the shape of the urethral sphincter. According to one aspect, the concave portion includes an active electrode length, or stimulation length, defined by the total longitudinal span of the electrodes along the elongate body. In one embodiment, the stimulation length may range to cover a surface area of the concave portion, from between 2 cm to 5 cm. As described in more detail below, in some embodiments the housing may include flexible features, and/or be formed of flexible materials, to enable the TSD to expand to conform within the vaginal canal when deployed within a patient.

The elongate body further includes a proximal introducer portion 205 extending distally from the concave portion, sized such that when the device is introduced into the vaginal canal the concave portion 204 engages the mid-urethra. The transvaginal device or stimulator may be anteriorly concave-shaped with a curvature capable of fitting urethra anatomy variations from 5 mm to 11 mm. Electrodes may be positioned on the surface such that they are between 0° and 45° relative to a longitudinal axis of the concave surface; In one exemplary embodiment, said curvature produces an electrode angle of between 15° and 20°, or between 18° relative to the horizontal reference, therefore hugging and compressing anteriorly up against the urethra.

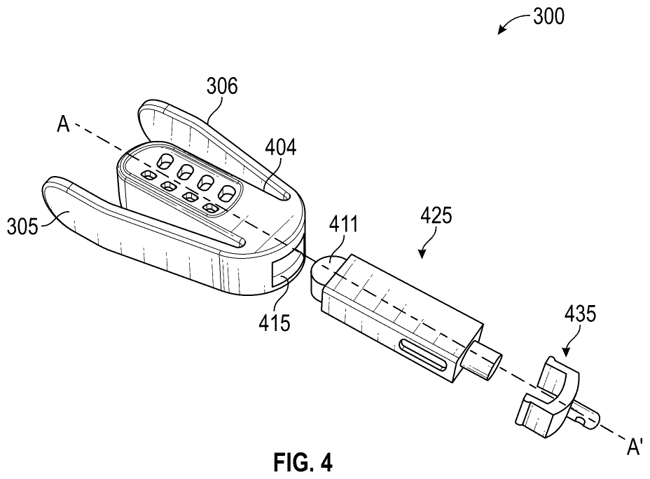

Bi-polar electrode pairs 206a-209a and 206b-209b are disposed upon the concave portion 204 of device 200, forming a stimulation region. A cap 210 (described in more detail in FIG. 4) may provide a seal between the external environment and internal circuitry within the TSD 200. The transvaginal stimulation device 200 is configured to be introduced into the vaginal cavity of the patient. The concave surface 204 and electrodes contact the anterior vaginal wall adjacent to the urethral sphincter and/or the levator ani muscles to deliver electrical stimulation energy through the anterior wall of the vagina towards the middle region of the urethra to target the mid-urethral striated sphincter muscle for stimulation.

It is appreciated that the concepts disclosed herein may be extended such that electrodes are disposed at various locations along the surface of the device to support stimulation of various pelvic muscle groups depending upon the desired treatment plan. Although specific embodiments are disclosed, the invention is not limited to merely the placement of electrodes and sensors disclosed herein.

According to one embodiment, the muscle stimulation electrodes in the concave portion 204 of the stimulating region are configured between 0° and 45° relative to the horizontal reference of the TSD to provide a curvature compatible with a range of 5-11 mm in urethra diameter. The concave shape of the electrode array is one example of a securing mechanism, providing a hugging and compressive effect on the anterior wall of the vagina, ensuring stable positioning of the electrodes and consistent energy delivery to the targeted muscle region during stimulation therapy. In some embodiments, the concave-shaped electrode array is designed to enclose medially or radially against the urethra, ensuring that electrical stimulation is directed solely towards the mid-urethral sphincter, minimizing the stimulation of surrounding non-targeted tissues.

The one or more pairs (206a/206b-209a/209b), of electrical contacts 206a may be electrically arranged to deliver energy in different patterns. For example, the current embodiment presents a 4×2 electrode array including electrode pairs 206a/206b, 207a/207b, 208a/208b and 209a/209b, for which 34 permutations of the energy direction and location can be applied. As a result, effective treatment may be provided to a larger section of the patient population across a wide range of anatomical structures and biological responses.

Table I below depicts 17 energy stimulation plans for applying energy is a first bi-polar direction, and an additional 17 energy stimulation plans available when the polarity is inverted.

TABLE I

| Electrode | Mx(+)1 | Mx(+)2 | Mx(+)3 | Mx(+)4 | Mx(−)1 | Mx(−)2 | Mx(−)3 | Mx(−)4 |
|---|---|---|---|---|---|---|---|---|
| ID | (206a) | (207a) | (208a) | (209a) | (206b) | (207b) | (208b) | (209b) |
| 1 × 8 | 1 | 0 | 0 | 0 | 0 | 0 | 0 | 1 |
| 1 × 7 | 1 | 0 | 0 | 0 | 0 | 0 | 1 | 0 |
| 1 × 6 | 1 | 0 | 0 | 0 | 0 | 1 | 0 | 0 |
| 1 × 5 | 1 | 0 | 0 | 0 | 1 | 0 | 0 | 0 |
| 2 × 8 | 0 | 1 | 0 | 0 | 0 | 0 | 0 | 1 |
| 2 × 7 | 0 | 1 | 0 | 0 | 0 | 0 | 1 | 0 |
| 2 × 6 | 0 | 1 | 0 | 0 | 0 | 1 | 0 | 0 |
| 2 × 5 | 0 | 1 | 0 | 0 | 1 | 0 | 0 | 0 |
| 3 × 8 | 0 | 0 | 1 | 0 | 0 | 0 | 0 | 1 |
| 3 × 7 | 0 | 0 | 1 | 0 | 0 | 0 | 1 | 0 |
| 3 × 6 | 0 | 0 | 1 | 0 | 0 | 1 | 0 | 0 |
| 3 × 5 | 0 | 0 | 1 | 0 | 1 | 0 | 0 | 0 |
| 4 × 8 | 0 | 0 | 0 | 1 | 0 | 0 | 0 | 1 |
| 4 × 7 | 0 | 0 | 0 | 1 | 0 | 0 | 1 | 0 |
| 4 × 6 | 0 | 0 | 0 | 1 | 0 | 1 | 0 | 0 |
| 4 × 5 | 0 | 0 | 0 | 1 | 1 | 0 | 0 | 0 |
| Open | 1 | 1 | 1 | 1 | 1 | 1 | 1 | 1 |

The electrodes 206a, 206b may respectively be activated as an anode and a cathode, such that electrical stimulation energy is transmitted between the electrodes 206a, 206b in a bi-polar manner. Although the electrical stimulation energy may be delivered as monophasic electrical energy (i.e., the pulses are either negative (cathodic) or positive (anodic), it is preferred that the electrical stimulation energy is delivered as multi-phasic electrical energy, e.g., a series of biphasic pulses, with each biphasic pulse including a negative (cathodic) pulse (during a first phase) and a positive (anodic) pulse (during a second phase) to prevent direct current charge transfer through tissue, thereby avoiding electrode degradation and cell trauma. In some embodiments, the electrodes may be low impedance electrodes that are resistant to delamination.

During therapy, the TSD may deliver stimulation to the muscles at 50 Hz, with pulse duration of 200-700 μs, with 1:2 treatment to rest cycles (1-4 s treatment with 2-8 s off). The pulse duration of the stimulation may be between 200-700 μs. The device may deliver stimulation to the muscles with 1:2 treatment to rest cycles (1-4 s treatment with 2-8 s off) for 10 to 20 minutes. Maximum current density should preferably not exceed 13.3 mA/cm².

FIGS. 3A and 3B illustrate one embodiment of a transvaginal stimulation device 300 including securing mechanisms comprising wings 305, 306, configured to expand from a first, compressed configuration, illustrated in FIG. 3A, to a second expanded position, illustrated in FIG. 3B. In one embodiment, the wings 305, 306 may be coupled at a first end to the proximal end of the housing 320, although it is appreciated that the invention is not limited by the location of attachment of the wings to the housing.

In one embodiment, the transvaginal stimulating device 300 is configured with one or more collapsable wings 30 to 150 deg from the stimulating region 350 to provide a small insertion profile (e.g., less than 0.5″×0.9″, for example) while collapsed and provide lateral positioning forces against the vaginal wall when in the open position. In some embodiments the wings 305, 306 may be made at least in part of shape metal, spring steel, or a polymer. They may be coated in a soft material such as silicone or medical grade Thermoplastic Elastomers (TPE) such as thermoplastic polyurethane, SEBS (Styrene-Ethylene-Butylene-Styrene).

In some embodiments, wings may be manually compressed for insertion, and the mechanical wing features 305, 306 are configured to expand to an angle between 30° to 150° laterally, preventing the transvaginal device from rotational misplacements and ensuring adequate contact against the mid-urethral section. The wings can be configured to have an expanded width W with a ratio of between 1 and 2 times the width during insertion. Wings may be configured to provide a consistent force acting on the vaginal walls. Alternatively, the device could be constructed with one or more collapsable wings opposed from the stimulating region (for instance approximately 180 degrees). According to one aspect, inventors have realized that stretching the vaginal wall laterally pulls the urethra posteriorly, ensuring better contact against the electrode array

The mechanism for expanding the wings may be automatic or manual. For example, in embodiments where the wings are formed of a memory alloy, expansion may occur automatically after insertion as the memory alloy expands due to memory and/or temperature operation on the alloy. The memory shaped wings provide constant and lateral pressure to the vaginal wall. Alternatively, where the wings are operated using springs or linkage mechanisms, expansion may be controlled manually, using a switch or dial to control expansion operation. In some embodiments, an expansion mechanism that includes a manually adjustable spring-loaded system, allows the user or clinician to set the desired pressure level before insertion, ensuring appropriate contact with the anterior wall based on individual patient requirements. In some embodiments, a mechanical lever may be integrated into the device, configured with a spring-loaded or electrically controlled mechanism that exerts pressure against the anterior vaginal wall, thereby enhancing the concave curvature's contact with the urethral tissue to improve stimulation accuracy and efficacy

FIG. 4 is an exemplary exploded view of the transvaginal stimulation device 300 of FIGS. 3A and 3B, comprising a housing 404 having wings 305, 306 and a port 415. The port 415 is adapted to receive a reusable controller 425. The controller may comprise a rechargeable, reusable and interchangeable component. In one embodiment, the controller 425 comprises a connector 411 adapted to mate with a connector internal to the housing 404 that is coupled to electrodes and/or sensors of the device 300. As discussed in more detail below, the controller 425 may house a flexible printed circuit board (not shown), for controlling electrode stimulation, processing sensor data and enabling communication between external users (physicians, patients) and the device. A cap 435 may be placed over the end of the controller 425 to protect the controller from damage during use.

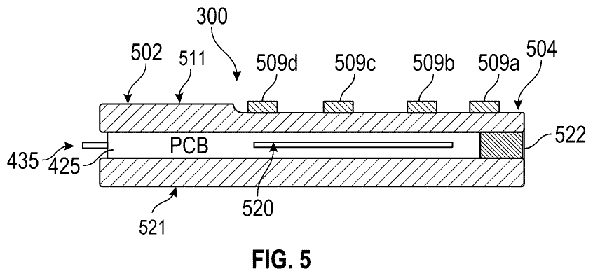

FIG. 5 is a cross-section view of the TSD 300 taken along line A-A′ of FIG. 4. The device 300 includes a housing 502, having a first surface 511 including a concave portion 504, and a second surface 521 opposite the first surface. The controller 425, shown inserted into port 411, engages connector 522, thereby connecting the electrodes (509a-509d) to processing circuitry within the controller 425 to control actuation of the electrodes. In some embodiments, the connector 522 further couples sensor circuitry provided by the housing to the processing circuitry.

A printed circuit board 520 is shown disposed within the controller 425. The printed circuit board (PCB) may be a flexible printed circuit board, such as a multi-layer flexible PCB, double access/back-bared flexible PCB, polymer thick film flexible PCB or the like. The PCB may comprise several inputs capable of taking information from the device's environment. The PCB may comprise several outputs capable of directing energy in different patterns through one or more electrical contacts. In other embodiments, the PCB may be formed of a rigid or semi-rigid material, and the invention is not limited by the resiliency of the PCB.

In some embodiments, the controller 425 may include an I/O interface programmed for external communication with a physician or patient. The communications may modify a selection of a therapy plan. For example, patient feedback regarding the intensity of the treatment may cause the controller to select a less invasive therapy plan for the patient.

In other embodiments, the transvaginal stimulation devices may include sensors, located within the controller and/or upon the body of the housing.

Sensors may be used to capture real-time biological information and/or positional information from the patient during treatment. This sensor data may be used to automatically dynamically modify at least one of a therapy plan or a device position within the vaginal canal.

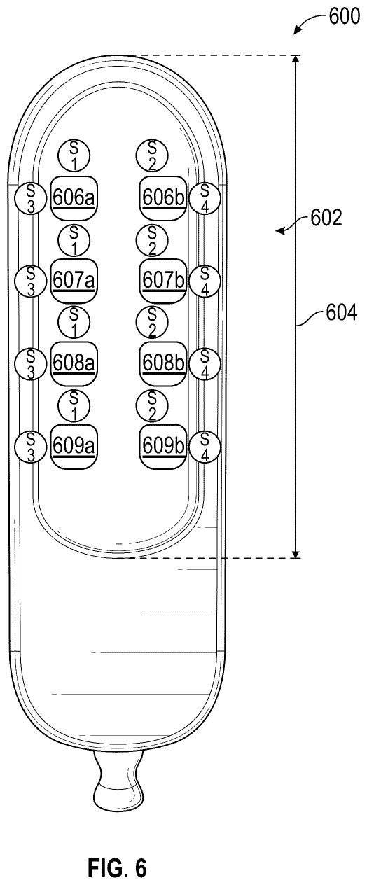

FIG. 6 illustrates an embodiment of a transvaginal stimulation device 600 having sensors S1-S4 disposed at various locations along the curved surface of housing 602. In FIG. 6, sensors S1-S4 are shown disposed near each of the pairs of electrodes 606a/606b-609a/609b and along the length of the curved portion 604, together forming a network of sensors selected from a group of sensors including impedance sensors, biomarker sensors, pressure sensors, accelerometers, gyroscopes, temperature sensors and infrared sensors. In some embodiments, the sensors may be low impedance sensors, as they are not subject to higher voltages/currents and delamination is not an issue.

The sensors continuously monitor biological measurements such as tissue impedance and adjust the stimulation parameters to maintain effective contact and stimulation delivery throughout the treatment session. The combined information from the sensors may be processed in real-time by machine learning models to dynamically modify stimulation therapy and device position based on real-time biological feedback and learned responses. As a result, the sensor network enables machine learning and artificial intelligence algorithms to deliver patient-specific muscle stimulation.

For example, the one or more electrical contact sensors may be used to measure the impedance of a section of the urethra, and/or to measure the average impedance along the axis of the transvaginal device. Additional smaller sensors may be located in between therapeutic electrodes to detect other biomarkers such as pH, urethra flow or electromyography (EMG) to continually analyze the anatomical response to the therapy. Although an arrangement is shown with groups of four types of sensors S1-S4 disposed about the body of the device 600, the present invention is not limited by the selection, placement or arrangement of sensors disclosed herein, as it is recognized that different types of sensors may benefit from placement at different locations depending upon the function they are intended to serve.

The sensors S1-S4 of TSD 600 may comprise one or more differential pressure sensors integrated into its structure to enhance diagnostic and therapeutic capabilities. The pressure sensor may be located on lateral aspects of the device (for example, at sensor locations S3 or S4) to increase information points. These pressure sensors are designed to detect and monitor subtle changes in pressure within the vaginal canal, providing real-time feedback on the device's position relative to the target tissue and the level of contact exerted against the anterior vaginal wall. The pressure differential sensors may indicate changes in muscle contraction over time, which compared to changes in impedance make it possible to approximate tissue response. By continuously measuring these pressure variations, the device can identify optimal positioning for effective stimulation of the mid-urethral sphincter muscle or other treatment location. For example, the pressure data can be fed into the device's internal processing system, which includes an analog block for signal conditioning and a digital block for data analysis. The embedded machine learning and artificial intelligence algorithms use this pressure information to dynamically adjust the stimulation parameters, such as energy intensity, contact pattern, and stimulation duration. This feedback-driven approach allows for the needed pressure application to ensure effective muscle contraction without causing discomfort or tissue damage.

The transvaginal device or stimulator may comprise one or more accelerometers embedded within its housing 602 to monitor orientation, movement, and positioning in real-time. Accelerometers detect changes in the device's spatial orientation and motion within the vaginal canal, providing important data for precise placement and stabilization of the stimulating electrodes against the target tissue. This information ensures that the device maintains consistent contact with the mid-urethral sphincter, optimizing the delivery of therapeutic electrical stimulation.

In addition to aiding in initial placement, accelerometers play a critical role in continuous monitoring during therapy sessions. They detect shifts in the device's position that may occur due to patient movement, muscle contractions, or changes in posture. The accelerometer data can be processed by the onboard analog and digital circuitry within the device's flexible PCB. This processing allows the device to make real-time adjustments to maintain proper orientation, ensuring the electrodes remain correctly aligned with the target area to deliver effective stimulation to the target treatment site. The conforming/flexible assembly allows the surface of the electrodes to accommodate anatomical variation. The integration of accelerometers also enhances the device's capability for adaptive therapy. As will be described in more detail below, using machine learning and artificial intelligence algorithms, the transvaginal device may analyze accelerometer data in conjunction with other sensor inputs discussed in the embodiment, to adjust stimulation parameters dynamically. For example, if the accelerometer detects a change in the device's tilt or rotation, the control system can modify the stimulation pattern to compensate for the altered contact angle, ensuring continuous and effective engagement of the urethral muscles.

The transvaginal device or stimulator may comprise one or more gyroscopes integrated within its housing 602 to provide detailed information about the device's rotational orientation. Gyroscopes measure angular velocity and rotational movements, enabling the device to maintain precise alignment within the vaginal canal. This precise alignment is important for ensuring that the stimulating electrodes consistently target the mid-urethral sphincter muscle, optimizing the delivery of electrical stimulation for effective therapy. The gyroscope data is processed by the device's flexible PCB, which includes both analog and digital circuitry. This processing allows the device to monitor its orientation in real-time, helping it maintain the desired positioning during therapy sessions. By continuously tracking any rotational changes, the gyroscopes assist in detecting device misalignment, such as twisting or tilting, which could affect the effectiveness of muscle stimulation. Additionally, gyroscopes work in tandem with accelerometers and other sensors to form a comprehensive sensing network. This network enables the device to adapt to the patient's anatomy and movements dynamically. The gyroscope data, when combined with information from accelerometers and differential pressure sensors, allows the onboard machine learning and artificial intelligence (AI) algorithms to make real-time adjustments to the stimulation patterns. For example, if the gyroscopes detect a rotational shift, the system can automatically adjust the activation pattern of the electrodes to ensure that stimulation remains focused on the intended tissue region. The inclusion of gyroscopes also enhances patient safety. During use, sudden changes in angular orientation detected by the gyroscopes can signal potential issues, such as device displacement or patient discomfort. In response, the device can trigger an alert or temporarily halt stimulation, providing an opportunity for repositioning or reassessment by the patient or healthcare provider. This safety mechanism ensures that stimulation is delivered only when the device is correctly aligned with the targeted muscle tissue. Furthermore, gyroscope data can be wirelessly transmitted to an external mobile device via Bluetooth or Wi-Fi, allowing healthcare professionals to monitor the device's performance and the patient's therapy progress. By reviewing rotational movement patterns and positional stability, clinicians can evaluate the quality of each therapy session and make informed adjustments to the treatment plan as needed.

The sensors S1-S4 of TSD 600 may comprise one or more temperature sensors positioned to monitor tissue temperature during therapy. Temperature sensing plays a critical role in ensuring patient safety and optimizing treatment efficacy, as it provides real-time feedback about the interaction between the device and the surrounding tissue. By continuously measuring the temperature at the contact site, the device can prevent potential thermal damage, maintain patient comfort, and ensure that the stimulation parameters remain within safe limits. The temperature sensors may be connected to a flexible PCB embedded in the device. This PCB may include analog circuitry for signal processing and digital components for data analysis. The analog circuitry filters and amplifies the temperature signals, which are then converted into digital data via one or more analog-to-digital converters (ADCs). The processed temperature data can be analyzed by the onboard machine learning and AI algorithms to make dynamic adjustments to the stimulation parameters, such as electrical intensity and duration. By incorporating one or more temperature sensors, the transvaginal device not only enhances the safety and comfort of the patient but also provides a dynamic and adaptive approach to therapy. This ensures that electrical stimulation is delivered within optimal thermal conditions, improving treatment outcomes and reducing the risk of adverse effects.

The sensors S1-S4 of the TSD 600 may comprise one or more infrared sensors to monitor tissue characteristics and biological responses during therapy. Infrared sensors are capable of non-invasive measurements, such as detecting blood oxygen levels, which are crucial for understanding tissue health and metabolic activity in the area being stimulated. By incorporating infrared sensors, the device can gather valuable biofeedback in real-time, enabling it to adaptively optimize stimulation parameters to enhance therapeutic outcomes. Infrared sensors work by emitting and detecting specific wavelengths of light that penetrate the tissue and interact with components such as blood and muscle fibers. This interaction provides data on factors like blood oxygenation, which can indicate the level of tissue perfusion and metabolic activity. The device's flexible PCB processes these signals through its analog circuitry before converting them into digital data via embedded ADCs. The digital block within the PCB then interprets this information using machine learning and AI algorithms to adjust the therapy dynamically. In practice, the integration of infrared sensors allows the device to adapt to the patient's unique tissue responses. For example, changes in oxygen saturation detected by the sensors can provide insights into how the tissue is responding to stimulation. Higher oxygen levels may indicate increased blood flow and muscle activity, signaling effective therapy. Conversely, reduced oxygenation might suggest the need to modify the stimulation pattern, intensity, or duration to achieve the desired therapeutic effects without overstressing the tissue. Furthermore, the infrared sensors contribute to the device's safety profile by monitoring tissue conditions continuously. If the sensors detect abnormalities in blood oxygenation, such as a significant drop indicating possible tissue hypoxia or an excessive increase suggesting overstimulation, the device can initiate corrective actions. These actions may include adjusting the electrical stimulation intensity, modifying the contact pattern of the electrodes, or pausing therapy to allow for tissue recovery. This level of monitoring helps prevents potential complications and ensures that the therapy remains within safe biological limits. In addition to providing immediate feedback, the data collected by the infrared sensors can be wirelessly transmitted to an external mobile device via Bluetooth or Wi-Fi. This transmission allows healthcare professionals to remotely monitor the patient's tissue responses during each therapy session. By analyzing this information, clinicians can make informed decisions about adjusting treatment protocols, tailoring the therapy to the patient's specific needs and enhancing overall efficacy.

The sensors of the sensor networks may be attached to the inputs of the inner flexible PCB, wherein the analog circuitry embedded in the PCB moves the signals through filtering and amplification, noise shaping and conversion to digital via one or more ADC components. The PCB may comprise a processor configured to execute a machine learning model trained to generate a therapy plan in response to received biological/positional input data, the therapy plan comprised of digital outputs that are multiplexed and sent to the one or more electrical contacts that enable the muscle stimulation energy to be delivered according to unique biological signatures of the patients. For example, by collecting temperature, pressure, angulation (gyroscope), change in positioning (accelerometer), oxygen level (infrared), and impedance, trained model can vary the amount of energy sent to the electrical contacts. It can vary the pattern of active electrical contacts based on areas that need more stimulation. It can indicate whether the stimulation has reached a saturation or muscular fatigue level, for example by monitoring change in impedance measurements,

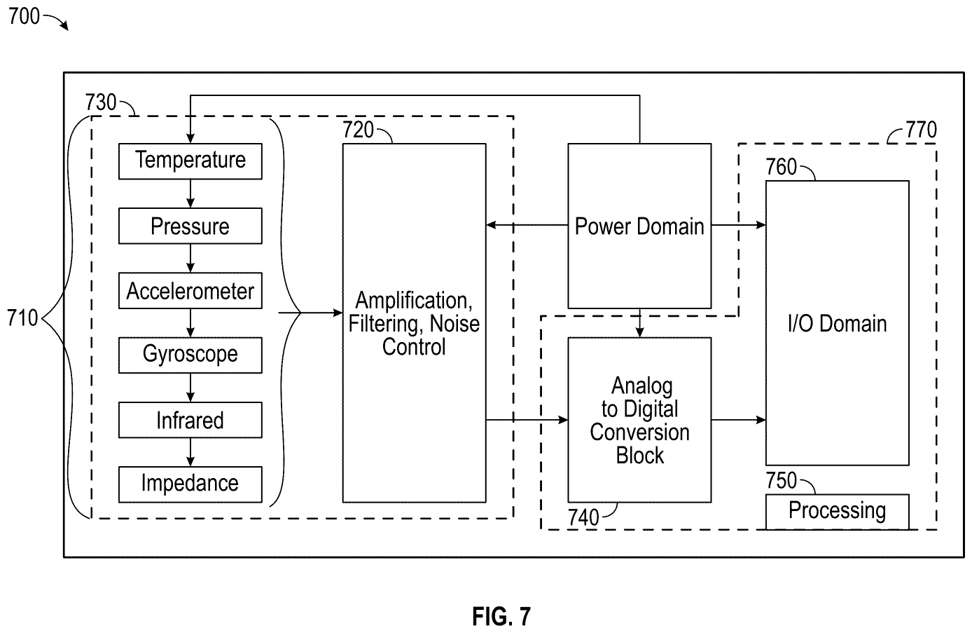

FIG. 7 shows a block diagram of the high-level functional blocks of the PCB involved in acquiring analog signals from the different sensors and using an amplification, filtering and digital conversion chain to process the signals so the digitized information can be sent wirelessly to a computer or mobile device.

Exemplary blocks of an exemplary PCB 700 are shown for the purposes of describing the exchange of data between the analog and digital domains. The PCB 700 may comprise a network of sensors 710 (e.g., temperature sensors, infrared sensors, pressure sensors, flow sensors, etc.) which are configured to receive biological and position information. The received analog signals are amplified, filtered and noise controlled via block 720 as part of an analog block 730. Biological information and positional data from the gyroscopes and accelerometers are processed and passed onto the analog to digital converter 740 of digital block 770. The analog to digital (A/D) conversion block 740 digitizes analog biological and positional information. The PCB digital block 770 may further include processing logic 750 configured to use predictive models, including but not limited to models that implement artificial intelligence techniques to interpret biological and positional information together with learned patient responses in real-time for the purposes of modifying therapy plans, modifying TSD position to improve the placement and/or the securing of the TSD within the vaginal canal, and/or halting therapy for safety or other reasons.

For example, processed temperature data can be analyzed by one or more predictive models, some of which have been trained using machine learning and AI techniques to make dynamic adjustments to the stimulation parameters, such as electrical intensity and duration. Additionally, the use of temperature sensors allows the device to monitor physiological responses during muscle stimulation. For instance, temperature changes in the tissue can indicate increased blood flow or metabolic activity, providing indirect feedback on the effectiveness of the therapy. The trained AI models interpret these changes and adapt the stimulation patterns to optimize muscle activation without causing overheating or discomfort. This adaptability is particularly beneficial for tailoring therapy to individual patient needs, as it ensures that the device responds to unique tissue characteristics in real-time. The integration of temperature sensors also adds a layer of safety to the device's operation. If the sensors detect an abnormal rise in tissue temperature—potentially indicating excessive stimulation or improper placement—the device can immediately trigger a response. This response may include reducing the stimulation intensity, pausing the therapy session, or alerting the patient and clinician to reposition the device. By incorporating temperature monitoring into its functionality, the device can deliver targeted stimulation while minimizing the risk of thermal injury.

The digital block further includes an I/O interface 760. The transvaginal device or stimulator may be paired via closed network wi-fi or Bluetooth to enable communication with a mobile device for operation and data display, and the I/O interface 760 may be programmed to support communication via such protocols, thereby allowing transmission of sensor data including biological responses to healthcare professionals for remote monitoring of the patient's tissue responses during each therapy session. By analyzing this information, clinicians can make informed decisions about adjusting treatment protocols, tailoring the therapy to the patient's specific needs and enhancing overall efficacy. For example, temperature, infrared, pressure or other data can be transmitted to an external mobile device via Bluetooth or Wi-Fi for real-time monitoring and assessment by healthcare professionals. This feature allows clinicians to observe temperature fluctuations during therapy, providing valuable insights into the patient's response to treatment. Based on this data, clinicians can make informed adjustments to therapy protocols, ensuring that each session is as safe and effective as possible, tailoring the therapy to the patient's specific needs and enhancing overall efficacy.

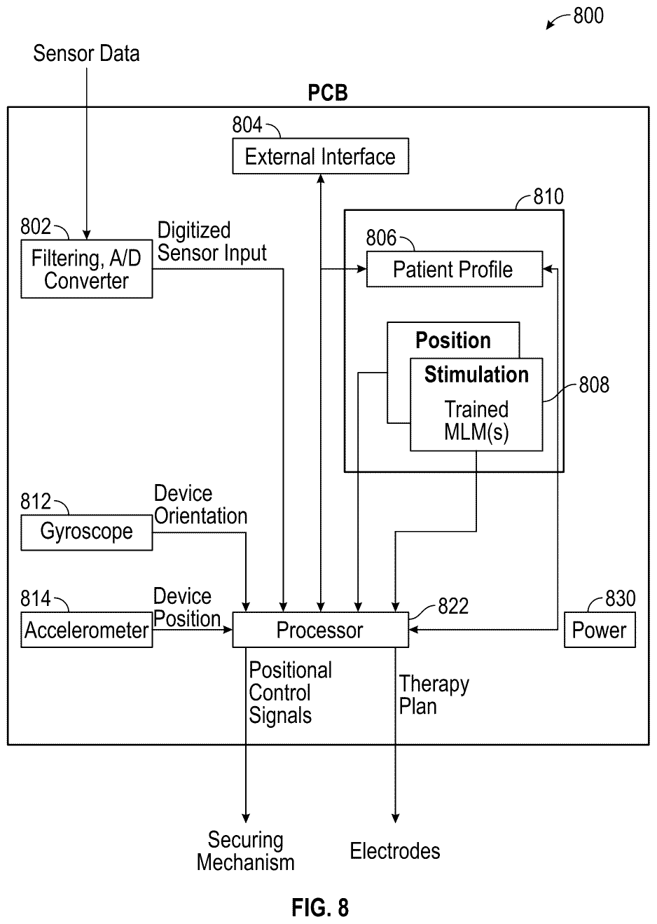

Referring now to FIG. 8, a block diagram illustrating exemplary functional blocks that may be provided on a PCB for use with a TSD as disclosed herein illustrates that the plurality of sensors (FIG. 7,711) may be attached to the inputs of an inner flexible PCB 800 via filtering and A/D conversion logic 802.

The PCB may comprise a memory 810 for storing program code corresponding to at least one previously trained machine learning model 808. Memory 810 may include non-transitory memory containing non-transitory instructions, such as a computer hard disk, random access memory (RAM), removable storage, or remote computer storage. In some aspects, memory 810 may be configured to store data and instructions, such as software programs. For example, memory 810 may be configured to store data and instructions. In some aspects, processor 822 may be configured to execute non-transitory instructions and/or programs stored on memory 810 to configure computing system provided on PCB 800 to perform operations of the disclosed systems and methods.

A processor 822 controls the operation of the TSD by executing the trained machine learning model (MLM) to produce a stimulation program for a therapy plan. Processor 822 may comprise a central processing unit (CPU), graphical processing unit (GPU), or similar micro-processor having one or more processing cores. Processor 822 may be formed from a Field Programmable Gate Array, programmed using the model stored in memory 810, or may include a programmable erasable programmable read-only memory (EPROM) that can be erased and reprogrammed with updated therapy plans.

Each therapy plan comprises a set of stimulation parameters (e.g., pulse amplitude (e.g., in the range of 6-120 mA), pulse frequency (e.g., in the range of 15 Hz-70 Hz), Pulse Duration: 200-700 μs, with 1:2 treatment to rest cycles (1-4 s treatment with 2-8 s off) of the electrical stimulation energy (i.e., the pulse train) output by the respective electrodes. For example, as shown in Table I above, the therapy plan may define the set of simulation parameters using the truth table to control the individual electrodes. The digital outputs of the therapy plan may be multiplexed and sent to the one or more electrical contacts that enable the muscle stimulation energy to be delivered according to unique biological signatures of the patients. For example, by collecting temperature, pressure, angulation (gyroscope 812), change in positioning (accelerometer 814), oxygen level (infrared), and impedance, the algorithm programmed into the PCB micro-processor can vary the amount of energy sent to the electrical contacts. It can vary the pattern of active electrical contacts based on areas that need more stimulation. It can indicate whether the stimulation has reached a saturation or muscular fatigue level.

In one embodiment, power module 830 comprises a rechargeable power circuitry including a battery, e.g., lithium-ion or lithium-ion polymer battery, and regulation circuitry (not shown). The battery outputs unregulated voltage to the regulation circuitry, which outputs regulated power to the electrodes, as well as the other components, of the transvaginal stimulation device. The power module 830 may be recharged using rectified AC power (or DC power converted from AC power). To recharge the power module 830 while the transvaginal stimulation device is disposed in the vaginal cavity of a patient, the transvaginal stimulation device may be connected via a port of the housing.

The PCB may further include an external interface 804 for transmitting sensor data to an external mobile device via Bluetooth or Wi-Fi for real-time monitoring and assessment by the patient and healthcare professionals. The external interface may further accept updated therapy plans, patient profile information 806 or patient input via the external interface 804.

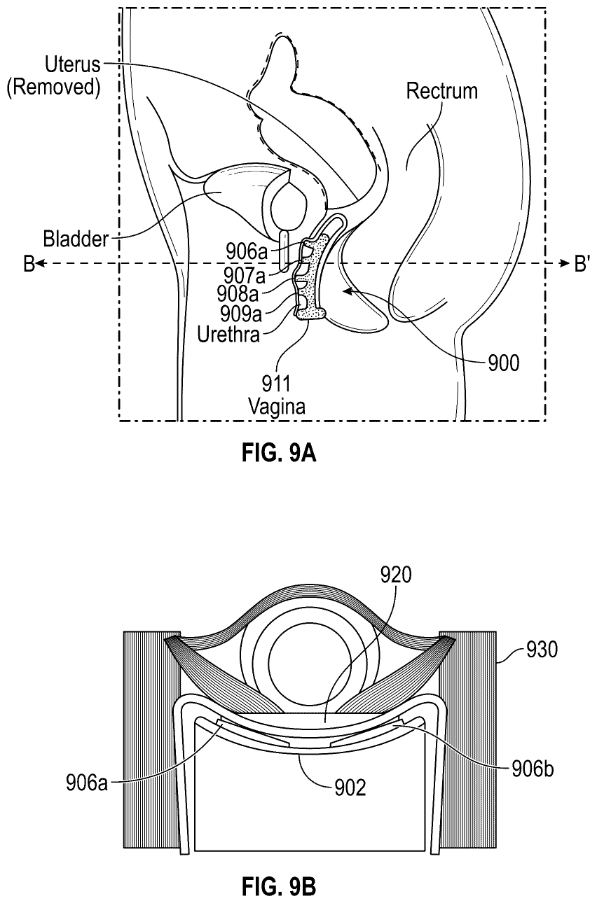

FIG. 9A is a cross-section view of female anatomy illustrating a transvaginal stimulation device 900 such as that disclosed herein having been inserted into a vaginal canal for therapy. The TSD 900 is constructed with a proximal positioning ring 911 located to provide a contact point against the meatus of the patient and provide consistent positioning of the electrodes 906a, 907a, 908a and 909a based on a distance (in the range of 1-4 cm) from the meatus. This positioning feature may contain a sensing (pressure, temperature, ultrasound, or impedance) positioning ring 911 to determine tissue contact is made. In some embodiments, this sensor may act as a switch to either turn on stimulation or turn off stimulation based on contact with the meatus. For example, using an impedance sensor to determine tissue contact with the meatus could allow the stimulation to be activated, and if tissue contact is lost during treatment the stimulation would stop until repositioned. In one embodiment, the contact point will be aligned longitudinally with the center of the electrodes to add both depth position and rotational position as the meatus is aligned with the urethra so centering on the urethra will provide orientation to the urethra for stimulation. Additionally, the loss or high changes in impedance would indicate poor device placement by disrupting the contact with one or more electrodes. The transvaginal device is configured so it detects changes above impedance threshold that indicate loss of placement and/or poor contact as well.

FIG. 9B is a cross-section of the anatomy of FIG. 9A taken along line B→B′. The TSD 900 is positioned such that as the transvaginal stimulation device is introduced into the vaginal cavity of the patient and for delivering electrical stimulation energy, the device 900 is directed with concave surface 902 including the electrodes 906a, 906b facing towards the anterior wall 930 of the vagina, thus limiting the delivery of electrical stimulation to the middle region of the urethra to target the mid-urethral striated sphincter muscle 920. In some embodiments, the anteriorly concave surface 902 may be designed with a curvature of approximately 5.5 mm, designed to conform to the anatomy of the mid-urethral anterior vaginal wall, allowing for optimal contact against the urethral tissue to target the mid-urethral sphincter muscle. The concave-shaped electrode array is designed to enclose medially or radially against the urethra, ensuring that electrical stimulation is directed solely towards the mid-urethral sphincter, minimizing the stimulation of surrounding non-targeted tissues.

It should be noted that the choice of curvature is design dependent; different curvatures may be desirable for treating different locations within the vaginal canal, such as the vaginal vault and/or the levator ani muscles.

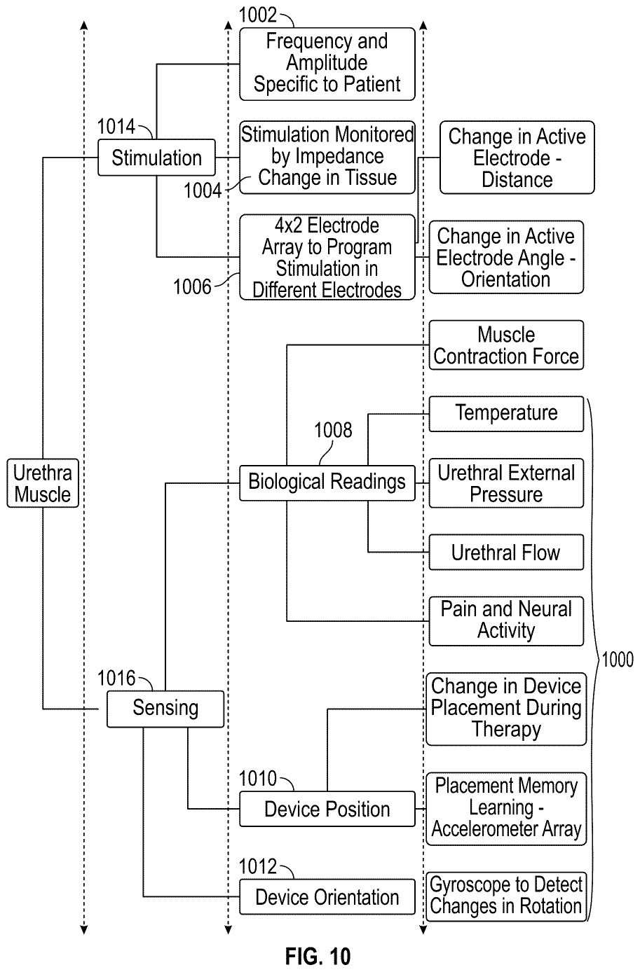

FIG. 10 is data flow diagram illustrating how sensed data may be used to improve device performance. A variety of biological and positional information may be provided from a plurality of sensor network data 1000 from a sensor network. In addition, although only one block for ‘Temperature’ is illustrated, it is appreciated that there may be multiple temperature sensors on a device, the temperature sensor data may include data from all temperature sensors in real-time. Predictive models may be trained to combine specific patient knowledge 1002, existing electrode array programs 1006 and impedance measurements 1004, together with existing biological readings 1008 and device position 1010 and orientation information 1012 to generate stimulations 1014 which result in further sensing of responses 1016. With such an arrangement, the ability of the TSD for sensing biofeedback from patients and delivering patient-specific muscle stimulation therapy to the mid-urethra section, will remain constant across a wide patient population.

FIGS. 3A and 3B disclosed a securing mechanism wherein compressible wings are provided to place lateral force upon vaginal walls upon insertion, urging the treatment region of the device towards the treatment location.

Alternative securing mechanisms will now be described. The alternative securing mechanisms may be used by TSD devices such as that of FIG. 6, which includes sensors. In such embodiments, the securing mechanism may be actuated using sensor feedback to control (drive, adapt) securing mechanisms for improved therapy. The securing mechanisms may also be used with TSD's such TSD 200 of FIG. 2, which does not include sensors. In such instances, the securing mechanisms may be actuated in response to patient and/or physician communications with the TSD controller.

A securing mechanism for dynamic response to real-time data may be implemented using a variety of techniques. The securing mechanism configured to expand device in the anterior, posterior or lateral directions, or circumferentially, in part or in whole, with the goal of securing the electrodes of the treatment portion of the device such that the concave portion of the device hugs the anterior wall of the vagina.

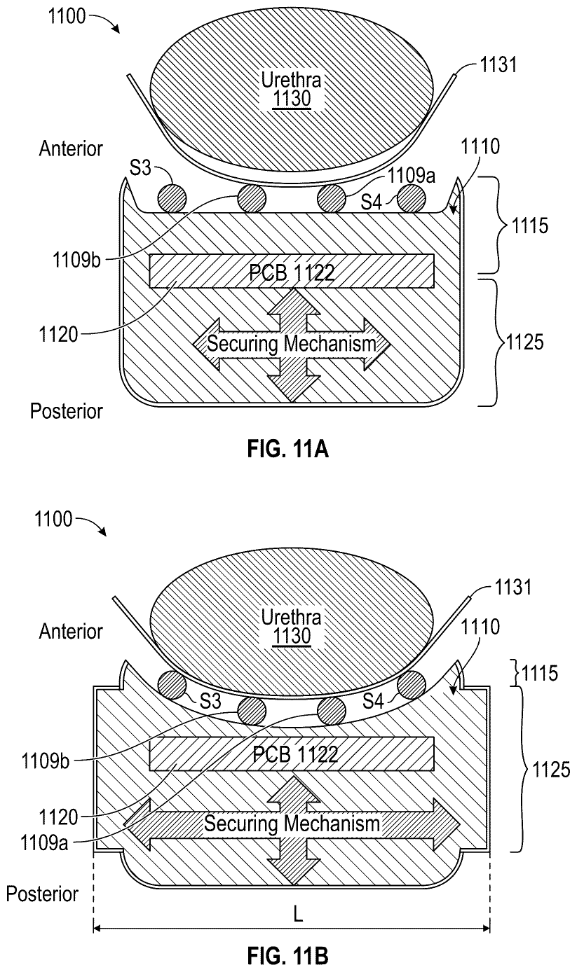

FIGS. 11A and 11B are cross sections of an exemplary generic TSD 1100 having alterative securing mechanisms. FIGS. 11A and 11B are taken along a craniocaudal perspective (top down). As described above, according to some embodiments, at least a portion 1115 of the housing 1120 of the transvaginal stimulation device 1100 is formed of a concave region 1110 extending longitudinally from the distal tip. Electrodes 1109a and 1109b are, in some embodiments, a pair of electrodes in an array of treatment electrodes which are activated according to a treatment plan to apply stimulation therapy to sphincter muscle 1131 of the urethra 1130. Sensors such as sensors S3 and S4 may be disposed about the body of the device 1100 to capture biological data in real-time as described above. Sensor data is processed by machine learning models on the printed circuit board 1122 which provides dynamic treatment plans for controlling delivery of therapeutic stimulation that is personalized to the patient.

The housing 1120 further includes a resilient portion 1125, formed of a resilient material such as thermoplastics and/or elastomers like silicon in 45A or lower durometer, allowing expansion and contraction of the TSD housing from a first compact profile to an expanded profile within the vaginal canal to secure the concave treatment portion 1110 of the TSD to the anterior vaginal wall, thereby improving contact between the sensors (S3, S4), electrodes (1109a, 1109b) and target tissue to optimize therapy.

FIG. 11B illustrates operation of an exemplary generic securing mechanism that expands the flexible portion in the anterior, posterior and lateral directions. According to one aspect, inventors have realized that stretching the vaginal wall laterally pulls the urethra posteriorly, ensuring better contact against the electrode array. Control of the securing mechanism may be executed to adjust the curvature of the concave electrode array based on patient-specific anatomy to maximize electrode-tissue contact, providing a personalized therapeutic approach for each patient. The concave shape of the electrode array is structured to deliver electrical stimulation in a focused manner, enhancing muscle contraction strength by conforming to the urethral tissue's natural shape and reducing electrical energy dispersion.

FIGS. 12A and 12B illustrate a transvaginal stimulation device 1210 comprising a securing mechanism comprising a spring-loaded mechanism 1222, electrodes 1209a, 1209b, sensors S3, S4, with the concave portion 1210 of the housing 1220, positioned toward the urethra 1230, in particular the urethra sphincter muscle 1231, towards the anterior vaginal wall. The device 1200 may comprise a resilient portion 1225 and a rigid or semi-rigid portion 1215. In some embodiments, the entire device 1200 may be made from a resilient material. In some embodiments, the spring-loaded mechanism 1222 automatically deploys upon insertion, expanding the posterior aspect of the device to press against the anterior vaginal wall, thereby improving the electrode-tissue interface for optimal muscle stimulation. Automatic deployment may be accomplished in response to received sensor data of real-time signals.

As shown in FIG. 12B, when the spring-loaded mechanism 1222 is expanding, it acts to conform the concave portion 1210 of device 1200 to the vaginal anatomy for optimal contact with the urethra 1230. The electrodes 1209a, 1209b, sensors S3 and S4 and concave surface 1210 are shown in detail, demonstrating the device's engagement with the target tissue. In some embodiments, the concave portion 1210 is rigid; in other embodiments, the concave portion 1210 is semi-rigid or flexible to permit minor or major deformity of the concave region 1210 to better engage the electrodes 1209a, 1209b with the target tissue 1231.

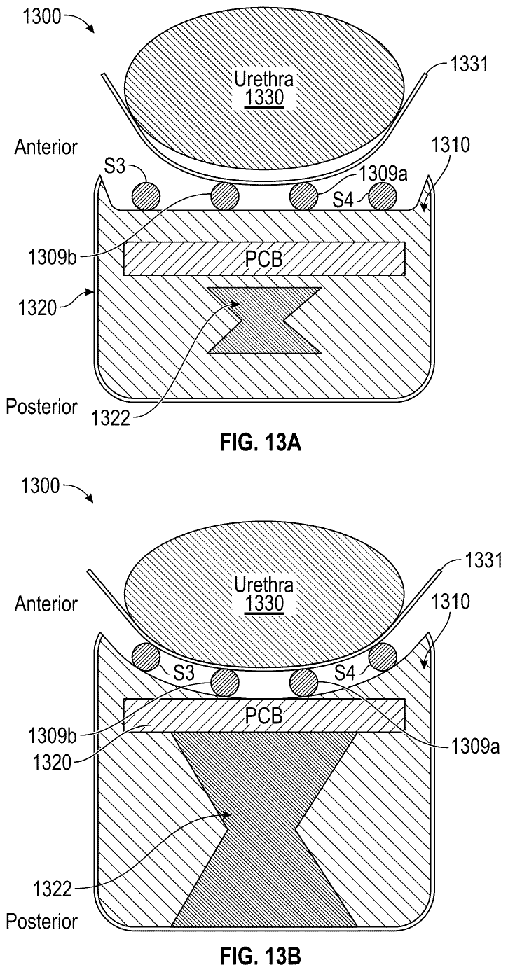

FIGS. 13A and 13B illustrate a transvaginal stimulation device 1300 comprising a securing mechanism comprising a 4-link mechanism 1322. The device 1300 further includes electrodes 1309a, 1309b, sensors S3, S4, with the concave portion 1310 of the housing 1320, positioned toward the urethra 1330, in particular the urethra sphincter muscle 1331, towards the anterior vaginal wall. At least a portion of the housing 1320 comprises a flexible, resilient material enabling better engagement of the device 1300 within the vaginal canal when the mechanism 1322 is in an expanded position.

The securing mechanism 1322 may include an array of miniature linear servo motors that actuate a 4-linkage assembly, allowing for controlled, precise expansion of the posterior aspect of the device to provide patient-specific pressure against the anterior vaginal wall.

FIG. 13B illustrates the transvaginal device 1300 in its expanded state, illustrating how its expansion may secure the device 1300 within the vaginal canal, better enabling contact between the electrodes 1309a, 1309b and sensors S3 and S4 on the concave surface 1310 and lateral edges of the device with the target tissue 1331.

FIGS. 14A and 14B illustrate a transvaginal stimulation device 1400 comprising a securing mechanism comprising a balloon 1422. The device 1400 may further include electrodes and sensors S3, S4 (as previously depicted with FIGS. 11A, 11B, 12A, 12B, 13A, and 13B), with the concave portion 1410 of the housing positioned toward the urethra sphincter muscle 1411 within the anterior vaginal wall. At least a portion of the housing comprises a flexible, resilient material enhancing the securing of the device 1400 within the vaginal canal when the balloon mechanism 1422 is in an expanded position.

FIG. 14B illustrates the transvaginal device 1400 with the balloon 1422 in its expanded state, illustrating how its expansion may secure the device 1400 within the vaginal canal at the treatment location (in region between arrows 1406) comprising the urethra 1430.

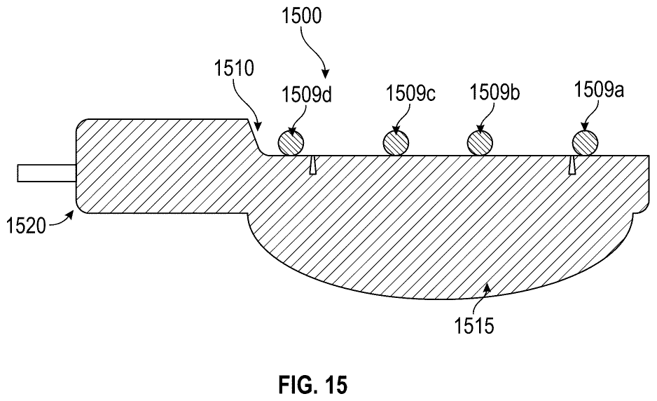

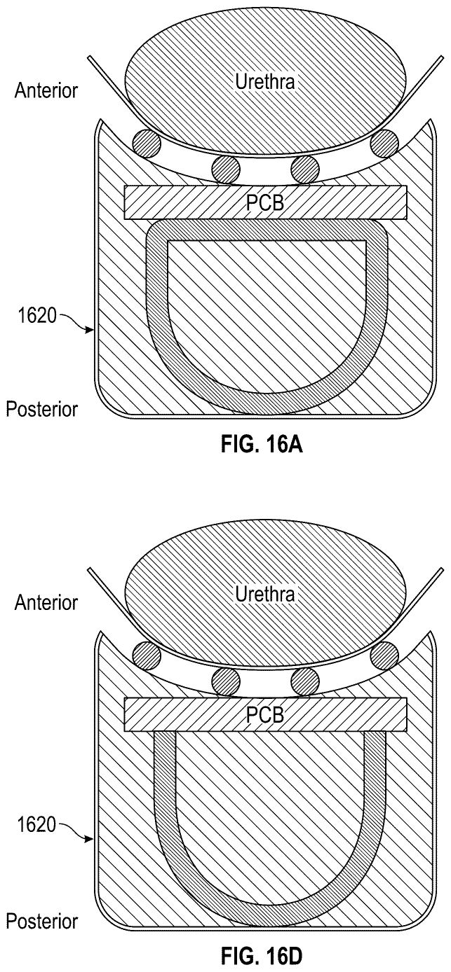

FIG. 15 is a side view of a transvaginal stimulation device 1500 when the securing mechanisms of any of FIGS. 11-14 have may be incorporated to expand the flexible portion 1515 of the housing 1520, to cause the electrodes 1509a-1509d of the concave surface 1510 to better engage with target tissue. In some embodiments, the housing incorporates a balloon as a posterior expansion mechanism coated with a soft, medical grade material, minimizing discomfort during deployment and providing a gentle yet secure grip against the vaginal walls to stabilize the device during therapy.

It is appreciated that although a balloon mechanism shown in FIGS. 14A, 14B and 15 may a general ‘D’ shaped cross-section, the present invention is not limited by the shape of the expansion balloon. Referring now to FIGS. 16A-16D, it is envisioned that expansion balloons of various configurations, such as ‘U’ shaped, ‘X’ shaped, D shaped and barbell shaped may be disposed within the housing 1620, with its planar surface fixedly attached to either or both the posterior or anterior inner walls of the housing. It is appreciated that the ability to provide balloons having different physical shapes provide different anterior, posterior and lateral forces when expanded that may be advantageous for securing the transvaginal stimulation device within the vaginal canal. In another embodiment, the transvaginal device is constructed with an expandable body region located under a non-expandable stimulating region.

For example, the expandable body region may be configured to provide radial expansion in a range of approximately 10-60 deg, while not expanding the stimulating region, thus providing pressure between the stimulating region and vaginal wall. The cross-section of the balloon may be constructed to take different shapes, such as a D (FIG. 16A) (with the stimulating region on the flat as shown in FIGS. 14A, 14B), U (FIG. 16B, with the stimulating region in the concave portion, X (FIG. 16C with the stimulating region between the legs), or other profiles such as a barbell depth profile FIG. 16D. It will be appreciated that the balloon and stimulating region may be formed of the same material (e.g. silicone), or that the different portions may be formed of different materials (e.g. expanding portion of flexible silicone and the stimulating region of medical grade plastic). Various materials can be used to inflate the balloon, such as complaint foam or gel in compositions that provide a comfortable fit. Polyurethane, silicon, polyvinyl alcohols (PVA), poly (N-isopropylacrylamide) (PNIPAM) hydrogel foams are examples of said materials.

According to another aspect of the invention, the transvaginal stimulation device may further include additional features integral with the housing to assist with positioning the device for treatment. These features may be used alone or in conjunction with any of the placement mechanisms disclosed above.

For example, referring now to FIG. 17, the body of the transvaginal stimulation device 1700 is formed with an anterior lip 1740 and a posterior lip 1750, each extending generally vertically in opposing directions from a horizontal plane defined by the longitudinal access of the device 1700. The lateral view of FIG. 17 highlights how the anterior flexible lip prevents over-insertion into the vaginal canal, and the posterior lip prevents against rotation and tilting of the device. device. As discussed above, in some embodiments, a positioning ring 1760 may be disposed between the anterior lip and posterior lip and may be configured to selectively enable treatment in response to contact between the ring and the patient.

In some embodiments, the transvaginal device or stimulator is constructed with a distal positioning ring 1760 located to provide a contact point against the meatus of the patient and provide consistent positioning of the electrodes based on a distance (in the range of 1-4 cm) from the meatus. This positioning feature may contain a sensing (pressure, temperature, ultrasound, or impedance) portion to determine tissue contact is made. In some embodiments, this sensor may act as a switch to either turn on stimulation or turn off stimulation based on contact with the meatus. For example, using an impedance sensor to determine tissue contact with the meatus could allow the stimulation to be activated, and if tissue contact is lost during treatment the stimulation would stop until repositioned. In one embodiment, the contact point will be aligned longitudinally with the center of the electrodes to add both depth position and rotational position as the meatus is aligned with the urethra so centering on the urethra will provide orientation to the urethra for stimulation. Additionally, the loss or high changes in impedance would indicate poor device placement by disrupting the contact with one or more electrodes. The transvaginal device is configured so it detects changes above impedance threshold that indicate loss of placement and/or poor contact as well.

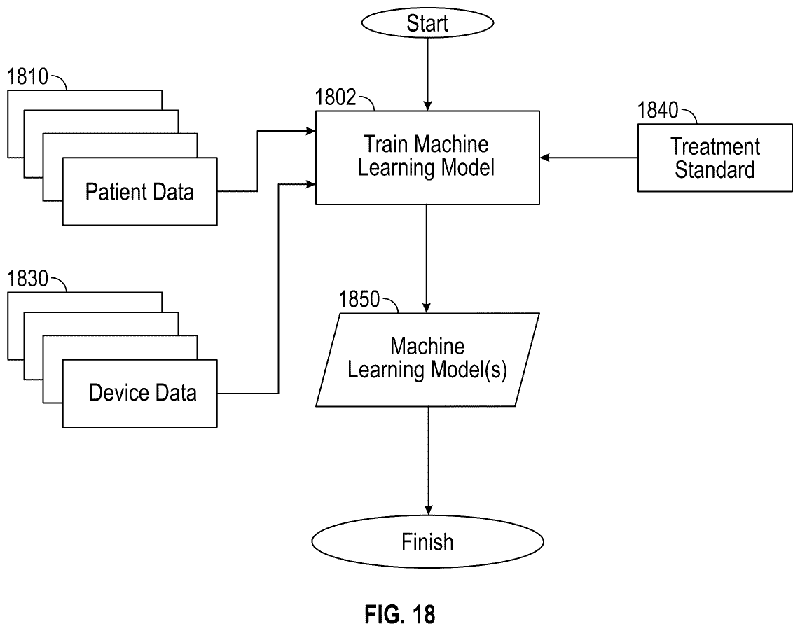

FIG. 18 depict flow diagrams illustrating exemplary operations that may be performed to train a machine learning model for use in transvaginal stimulation devices such as those disclosed herein. At operation 1802, a machine learning model for controlling a transvaginal stimulation device may be generated using a variety of forms of machine learning, including supervised learning, unsupervised learning, semi-supervised and reinforcement learning. For example, supervised learning may be performed by collecting historical patient data (including patient biological characteristics, patient reactions) and standard of care information. Systems such as expert learning systems may use data from previous therapy sessions administered by experts in the field to identify a treatment plan. Unsupervised, semi-supervised systems and/or reinforcement learning, or other technique, may use patient feedback data from the treatment to identify a patient-specific response to therapies. The learning involves collecting patient data comprised of digitized biological signal information. The patient data 1810 may be analyzed by machine learning algorithms to expose features of the dataset; i.e., isolating specific signals or patterns relevant to the patient's treatment. A neural network, such as a convolutional neural network (CNN) is taught to recognize the features and their relationship to a desired outcome based on treatment standard information 1840. Device data 1830, related to the electrical arrangement and capabilities of particular transvaginal stimulation device, is also considered when teaching the model how to achieve the desired outcome. A patient-specific, device specific machine learning model 1850 may then be generated for use in the transvaginal stimulation device.

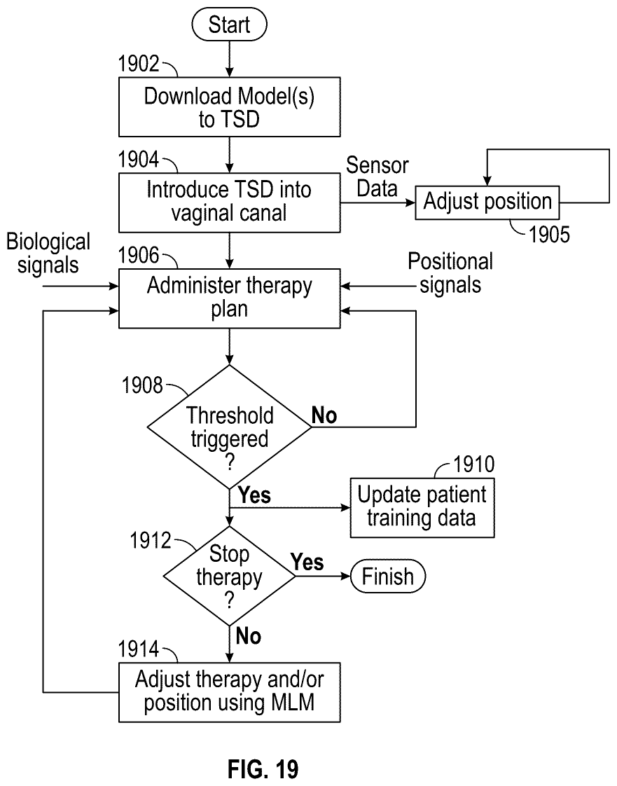

FIG. 19 is a flow diagram illustrating an exemplary method for using a transvaginal stimulation device such as that disclosed herein. At operation 1902, a machine learning model may be downloaded to the TSD, either via a wireless or Bluetooth communication with the device or by inserting the flexible PCB, with a pre-loaded machine learning model, into the TSD. AT operation 1904, the device may be inserted into the vaginal canal of the patient. Once the device is inserted, sensor is continually introduced to the machine learning model. At operation 1905, the securing mechanism operates to continually adjust the position of the of the curved portion of the TSD to conform to the anatomy of the vaginal wall, allowing for optimal contact against the urethral tissue to target the mid-urethral sphincter muscle and ensure that electrical stimulation is directed solely towards the mid-urethral sphincter, minimizing the stimulation of surrounding non-targeted tissues.

At operation 1906 the selected therapy plan is administered to the patient. According to one aspect, as described above with regard to Table I, the ‘therapy plan’ may in some embodiments include 34 potential permutations of energy direction and location, covering a larger patient population, and easily enabling on the fly therapy modification depending upon the response of the individual patient.

At operation 1908, one or more thresholds is evaluated to measure the effectiveness of the selected plan. The thresholds may be set based on individual sensor information or the learned relationships between multiple different types of sensor data. For example, as described above, continuously measuring these pressure variations, the device can identify optimal positioning for effective stimulation of the mid-urethral sphincter muscle. By collecting temperature, pressure, angulation (gyroscope), change in positioning (accelerometer), oxygen level (infrared), and impedance; the algorithm programmed into the PCB micro-processor can vary the amount of energy sent to the electrical contacts. During use, sudden changes in angular orientation detected by the gyroscopes can signal potential issues, such as device displacement or patient discomfort. In response, the device can trigger an alert or temporarily halt stimulation, providing an opportunity for repositioning or reassessment by the patient or healthcare provider. This safety mechanism ensures that stimulation is delivered only when the device is correctly aligned with the targeted muscle tissue. At operation 1912, the process determines whether the trigger indicates a shutdown, or simply an altered therapy plan.

Should a trigger condition occur that does not indicate a stop in therapy, at operation 1914, the patient information may be used to alter the therapy plan, for example, by selecting another treatment of the potential 34 permutations, adjusting the position of the device, or a combination thereof. In some embodiments, the updated patient training data 1910 may be forwarded externally from the device to further train the machine learning models with patient-specific data. The process continues until, at operation 1912 it is determined that therapy is concluded.

Accordingly, several embodiments of a transvaginal stimulation device have been disclosed, the embodiments incorporating various securing mechanisms configured to maintain secure engagement between the treatment portion of a transvaginal stimulation device and target tissue to increase the efficacy of therapeutic stimulation treatment. The securing mechanisms may be provided together with an embodiment including a concave treatment area, configured with an array of electrodes such that when the concave portion of the device hugs the tissue to be treated. Electrodes are preferably arranged to maximize the contact between electrode pairs and target tissue. A flexible housing increases the ability of the device to conform to the vaginal canal to maximize contact. The securing mechanisms may be manually or automatically controlled. Automatic control may be facilitated through the use of predictive models that have been trained using patient data to manage therapy and positioning of the device for maintain the device securely at its treatment location. In some embodiments, therapy may be automatically, dynamically managed using machine learning models to process real-time biological and positional data.

Other embodiments will be apparent to those skilled in the art from consideration of the specification and practice of the disclosed embodiments disclosed herein. It is intended that the specification and examples be considered as exemplary only, with a true scope and spirit of the disclosed embodiments being indicated by the following claims. Furthermore, although aspects of the disclosed embodiments are described as being associated with data stored in memory and other tangible computer-readable storage mediums, one skilled in the art will appreciate that these aspects can also be stored on and executed from many types of tangible computer-readable media, such as secondary storage devices, like hard disks, floppy disks, or CD-ROM, or other forms of RAM or read-only memory (ROM). Accordingly, the disclosed embodiments are not limited to the above described examples, but instead is defined by the appended claims in light of their full scope of equivalents.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as non-exclusive. Further, the operations of the disclosed methods can be modified in any manner, including by reordering operations or inserting or deleting operations. It is intended, therefore, that the specification and examples be considered as example only, with a true scope and spirit being indicated by the following claims and their full scope of equivalents.

Example 1 provides a device including an elongate body extending axially from a proximal end to a distal end, the elongate body including a concave portion curved about a longitudinal axis of the elongate body; a plurality of electrode pairs, each pair disposed at locations on the concave portion of the elongate body, where each electrode of the pair is positioned laterally from, and on opposing sides of, the longitudinal axis of the elongate body; and a securing mechanism that applies at least one of a lateral or an axial force to a wall of a vaginal canal to conform the concave portion of the elongate body about a urethra treatment location to secure the electrodes to selected stimulation points of the urethra treatment location; where applying force to the wall of the vaginal canal increases and/or maintains a contact between the plurality of electrode pairs and the urethra treatment location.

Example 2 provides the device of example 1, where at least one of the electrodes are disposed at locations of the concave portion in a range of between 0 and 45 degrees offset from the longitudinal axis of the elongate body.

Example 3 provides the device of example 1 or 2, where the plurality of electrode pairs are programmed to support at least thirty-four (34) muscle stimulation therapy plans, and a current density of the electrodes ranges between 10 and 20 mA/cm<sup>2</sup>.

Example 4 provides the device of any one of examples 1-3, further including a wireless communication interface to receive a selection of a muscle stimulation therapy plans from a plurality of muscle stimulation therapy plans.

Example 5 provides the device of any one of examples 1-4, where the concave portion has a diameter between 5 mm and 10 mm, an active electrode length between 2 and 5 cm, and the urethra treatment location includes a urethra.

Example 6 provides the device of any one of examples 1-5, where the securing mechanism includes a pair of longitudinal wing extensions, coupled to the proximal end of the elongate body and extending towards the distal end of the elongate body, where the pair of longitudinal wing extensions include a memory shaped alloy compressible to align the pair of longitudinal wing extensions with the elongate body for insertion into the vaginal canal, and releasable following insertion to open the pair of longitudinal wing extensions to apply lateral forces to a vaginal wall to pull the urethra treatment location towards the concave portion of the elongate body and to secure the device within the vaginal canal.

Example 7 provides the device of any one of examples 1-6, where the securing mechanism includes an anterior lip extending from the proximal end of the elongate body, the anterior lip directed toward an anterior vaginal wall when inserted into the vaginal canal and to preclude insertion of the concave portion of the elongate body past the urethra treatment location; and a posterior lip extending from the proximal end of the elongate body, the posterior lip directed towards a posterior vaginal wall and configured to reduce rotation of the elongate body following placement of the elongate body within the vaginal canal.

Example 8 provides the device of any one of examples 1-7, where the securing mechanism is controlled manually, wirelessly, or via a processor within the device.

Example 9 provides the device of any one of examples 1-8, where the securing mechanism includes a balloon.

Example 10 provides the device of any one of examples 1-9, where the securing mechanism includes a mechanical lever including at least one of a spring-loaded or electrically controlled mechanism that exerts pressure laterally or axially within the vaginal canal.

Example 11 provides a device including a disposable elongate body extending axially from a proximal end to a distal end, the disposable elongate body including a concave portion curved about a longitudinal axis of the disposable elongate body, the concave portion ranging in length between 2 and 5 cm; a plurality of electrode pairs, each pair disposed at locations on the concave portion of the disposable elongate body; a securing mechanism configured to conform the concave portion of the disposable elongate body about a treatment location of a urethra to secure the plurality of electrode pairs at selected stimulation points of the treatment location, and to provide fixed contact between the plurality of electrode pairs and the treatment location; and a reusable controller, coupled to the plurality of electrode pairs, the reusable controller activating individual electrode pairs within the plurality of electrode pairs according to a therapy plan for a patient.

Example 12 provides the device of example 11, where the plurality of electrode pairs form an electrode array having at least one row and at least two columns, and at least a subset of the electrodes is disposed at locations on a concave surface of the disposable elongate body that are between 0 and 45 degrees offset from the longitudinal axis of the disposable elongate body.

Example 13 provides the device of example 11 or 12, where the plurality of electrode pairs are programmed to support at least thirty-four (34) muscle stimulation therapy plans identifying pulse durations in a range of 200-700 microseconds, and pulse frequencies in a further range of 15-70 Hz, and a current density of the electrodes ranges between 10 and 20 mA/cm<sup>2</sup>.

Example 14 provides the device of any one of examples 11-13, where the reusable controller: updates a treatment plan in response to feedback; and modifies an order of activation of the electrodes in accordance with the updated treatment plan.

Example 15 provides the device of example 14, where the feedback may include at least one or more of: external feedback and internal feedback, the external feedback including patient feedback and the internal feedback including trigger detection.

Example 16 provides a device including an elongate body extending axially from a proximal end to a distal end, the elongate body including a concave portion curved about a longitudinal axis of the elongate body; a plurality of electrode pairs, each pair disposed at locations on the concave portion of the elongate body; a securing mechanism that applies at least one of a lateral or an axial force to a wall of a vaginal canal to conform the concave portion of the elongate body about a treatment location; at least one sensor to receive, in real-time, sensor data including at least one of patient biological data and positional data; and a controller, coupled to the plurality of electrode pairs and the at least one sensor, the controller to: actuate the securing mechanism in response to received sensor data; and activate individual electrode pairs within the plurality of electrode pairs using a therapy plan based on the sensor data.

Example 17 provides the device of example 16, where the concave portion of the elongate portion includes a curvature that is selected to hug and compress an anterior vaginal wall adjacent to a urethral sphincter.

Example 18 provides the device of example 16 or 17, where the at least one sensor include a biological sensor selected from a group including an impedance sensor, a muscle response sensor (EMG), a temperature sensor, a blood oxygen sensor, and a flow sensor, and the at least one sensor are to capture biological data in real-time.

Example 19 provides the device of any one of examples 16-18, where the at least one sensor is disposed on at least one of the distal portion of a housing of the elongate body, the concave portion of the housing, and/or within the housing of the elongate body.

Example 20 provides the device of any one of examples 16-19, where the at least one sensor is a positional sensor selected from a group including an accelerometer, and a gyroscope.

Example 21 provides the device of any one of examples 16-20, where the controller is removable from the elongate body.

Example 22 provides the device of any one of examples 16-21, where the securing mechanism is selected from a group of expandable mechanisms including memory alloy expandable mechanisms, mechanical expandable mechanisms, resilient expandable mechanisms, and fluid.

Example 23 provides the device of any one of examples 16-22, where the controller further includes a predictive model, trained to dynamically control at least one or more of the therapy plan, and a position of the device, in response to patient biological and positional data received in real-time.

Example 24 provides the device of example 23, the controller further including a communication unit configured to receive external communications, and where the predictive model uses the external communications to control at least one or more of the therapy plan, and a position of the device.

1. A device comprising:
an elongate body extending axially from a proximal end to a distal end, the elongate body comprising a concave portion curved about a longitudinal axis of the elongate body;
a plurality of electrode pairs, each pair disposed at locations on the concave portion of the elongate body, where each electrode of the pair is positioned laterally from, and on opposing sides of, the longitudinal axis of the elongate body; and
a securing mechanism that applies at least one of a lateral or an axial force to a wall of a vaginal canal to conform the concave portion of the elongate body about a urethra treatment location to secure the electrodes to selected stimulation points of the urethra treatment location;
wherein applying force to the wall of the vaginal canal increases and/or maintains a contact between the plurality of electrode pairs and the urethra treatment location.

2. The device of claim 1, wherein at least one of the electrodes are disposed at locations of the concave portion in a range of between 0 and 45 degrees offset from the longitudinal axis of the elongate body.

3. The device of claim 1, wherein the plurality of electrode pairs are programmed to support at least thirty-four (34) muscle stimulation therapy plans, and a current density of the electrodes ranges between 10 and 20 mA/cm².

4. The device of claim 1, further comprising a wireless communication interface to receive a selection of a muscle stimulation therapy plans from a plurality of muscle stimulation therapy plans.

5. The device of claim 1, wherein the concave portion has a diameter between 5 mm and 10 mm, an active electrode length between 2 and 5 cm, and the urethra treatment location includes a urethra.

6. The device of claim 1, wherein the securing mechanism comprises a pair of longitudinal wing extensions, coupled to the proximal end of the elongate body and extending towards the distal end of the elongate body, wherein the pair of longitudinal wing extensions comprise a memory shaped alloy compressible to align the pair of longitudinal wing extensions with the elongate body for insertion into the vaginal canal, and releasable following insertion to open the pair of longitudinal wing extensions to apply lateral forces to a vaginal wall to pull the urethra treatment location towards the concave portion of the elongate body and to secure the device within the vaginal canal.

7. The device of claim 1, wherein the securing mechanism comprises:
an anterior lip extending from the proximal end of the elongate body, the anterior lip directed toward an anterior vaginal wall when inserted into the vaginal canal and to preclude insertion of the concave portion of the elongate body past the urethra treatment location; and
a posterior lip extending from the proximal end of the elongate body, the posterior lip directed towards a posterior vaginal wall and configured to reduce rotation of the elongate body following placement of the elongate body within the vaginal canal.

8. The device of claim 1, wherein the securing mechanism is controlled manually, wirelessly, or via a processor within the device.

9. The device of claim 1, wherein the securing mechanism comprises a balloon.

10. The device of claim 1, wherein the securing mechanism comprises a mechanical lever comprising at least one of a spring-loaded or electrically controlled mechanism that exerts pressure laterally or axially within the vaginal canal.

11. A device comprising:
a disposable elongate body extending axially from a proximal end to a distal end, the disposable elongate body comprising a concave portion curved about a longitudinal axis of the disposable elongate body, the concave portion ranging in length between 2 and 5 cm;
a plurality of electrode pairs, each pair disposed at locations on the concave portion of the disposable elongate body;
a securing mechanism configured to conform the concave portion of the disposable elongate body about a treatment location of a urethra to secure the plurality of electrode pairs at selected stimulation points of the treatment location, and to provide fixed contact between the plurality of electrode pairs and the treatment location; and
a reusable controller, coupled to the plurality of electrode pairs, the reusable controller activating individual electrode pairs within the plurality of electrode pairs according to a therapy plan for a patient.

12. The device of claim 11, wherein the plurality of electrode pairs form an electrode array having at least one row and at least two columns, and at least a subset of the electrodes is disposed at locations on a concave surface of the disposable elongate body that are between 0 and 45 degrees offset from the longitudinal axis of the disposable elongate body.

13. The device of claim 11, wherein the plurality of electrode pairs are programmed to support at least thirty-four (34) muscle stimulation therapy plans identifying pulse durations in a range of 200-700 microseconds, and pulse frequencies in a further range of 15-70 Hz, and a current density of the electrodes ranges between 10 and 20 mA/cm².

14. The device of claim 11, wherein the reusable controller:
updates a treatment plan in response to feedback; and
modifies an order of activation of the electrodes in accordance with the updated treatment plan.

15. The device of claim 14 wherein the feedback may include at least one or more of: external feedback and internal feedback, the external feedback including patient feedback and the internal feedback including trigger detection.

16. A device comprising:
an elongate body extending axially from a proximal end to a distal end, the elongate body comprising a concave portion curved about a longitudinal axis of the elongate body;
a plurality of electrode pairs, each pair disposed at locations on the concave portion of the elongate body;
a securing mechanism that applies at least one of a lateral or an axial force to a wall of a vaginal canal to conform the concave portion of the elongate body about a treatment location;
at least one sensor to receive, in real-time, sensor data including at least one of patient biological data and positional data; and
a controller, coupled to the plurality of electrode pairs and the at least one sensor, the controller to:
actuate the securing mechanism in response to received sensor data; and
activate individual electrode pairs within the plurality of electrode pairs using a therapy plan based on the sensor data.

17. The device of claim 16, wherein the concave portion of the elongate portion comprises a curvature that is selected to hug and compress an anterior vaginal wall adjacent to a urethral sphincter.

18. The device of claim 16, wherein the at least one sensor comprise a biological sensor selected from a group including an impedance sensor, a muscle response sensor (EMG), a temperature sensor, a blood oxygen sensor, and a flow sensor, and the at least one sensor are to capture biological data in real-time.

19. The device of claim 16, wherein the at least one sensor is disposed on at least one of the distal portion of a housing of the elongate body, the concave portion of the housing, and/or within the housing of the elongate body.

20. The device of claim 16, wherein the at least one sensor is a positional sensor selected from a group including an accelerometer, and a gyroscope.

21. The device of claim 16, wherein the controller is removable from the elongate body.

22. The device of claim 16, wherein the securing mechanism is selected from a group of expandable mechanisms including memory alloy expandable mechanisms, mechanical expandable mechanisms, resilient expandable mechanisms, and fluid.

23. The device of claim 16, wherein the controller further includes a predictive model, trained to dynamically control at least one or more of the therapy plan, and a position of the device, in response to patient biological and positional data received in real-time.

24. The device of claim 23, the controller further comprising a communication unit configured to receive external communications, and wherein the predictive model uses the external communications to control at least one or more of the therapy plan, and a position of the device.
